(19) United States
(12) Patent Application Publication
(10) Pub. No.: US 2019/0129020 A1
(43) Pub. Date: May 2, 2019

(54) ULTRASOUND SYSTEM AND METHOD WITH ADAPTIVE OVERFLOW AND GAIN CONTROL

(71) Applicant: Industrial Technology Research Institute, Hsinchu (TW)

(72) Inventors: Yung-Shun Huang, Hsinchu (TW); Yuan-Mao Hung, Hsinchu (TW); Ming-Che Lin, Hsinchu (TW); Yi-Hsin Lin, Hsinchu (TW)

(21) Appl. No.: 15/853,829

(22) Filed: Dec. 24, 2017

(30) Foreign Application Priority Data
Oct. 31, 2017 (TW) 106137577

(51) Int. Cl. G01S 7/52; G01S 15/89; A61B 8/08

(52) U.S. Cl. CPC G01S 7/52033; G01S 15/8984; A61B 8/5215; A61B 8/488; G01S 7/52077

(57) ABSTRACT

An ultrasonic system and an ultrasonic method with adaptive overflow and gain control are provided. The present disclosure uses two-stage gain controls to improve the signal-to-noise ratio (SNR) and to achieve the correct analysis of results. The first stage adopts an analog gain, and monitors whether the signal may overflow or its signal strength is too weak, and adjusts the gain value as large as possible, without causing overflow in order to improve the SNR. The second stage adopts a digital gain. In the second stage, all the amplified gain-containing data adjusted in the first stage are received, and an amplified gain-containing data segment is sequentially retrieved in chronological order, and the amplified gain-containing data segment is converted into a same-gain data segment having the same gain. Finally, the same-gain data segment is provided to a Doppler signal analyzer for analysis.

### CROSS-REFERENCE TO RELATED APPLICATION

The present disclosure is based on, and claims priority from, Taiwan Application Number 106137577, filed Oct. 31, 2017, the disclosure of which is hereby incorporated by reference herein in its entirety.

### BACKGROUND

### 1. Technical Field

The present disclosure relates to control systems and methods for an ultrasound system, and more particularly, to an ultrasound system and method with adaptive overflow and gain control.

### 2. Description of Related Art

Medical ultrasound is an ultrasound-based medical imaging diagnostic technique. Doppler ultrasound, which belongs to the field of medical ultrasound, uses the Doppler Effect to determine whether a structure (generally a blood flow) is moving toward or away from the probe and calculate its relative velocity. By calculating the frequency drift of a portion of the sample volume, such as a jet flow over the heart valve, its direction and velocity can be determined and displayed. This is particularly useful for cardiovascular research and is necessary for other medical fields, such as retrograde blood flow in the diagnosis of arteries. Graphical display of Doppler information may use frequency spectrum Doppler, color Doppler or energy Doppler.

However, the filtering and gain control circuit in the existing Doppler ultrasound system needs to amplify the weak Doppler shift signal. If the gain value is adjusted too small, and when the Doppler shift signal is too small or too close to the subsequent noise levels, poor Signal-to-Noise Ratio (SNR) may be produced, which will not truly and completely reflect the Doppler shift signal representing the velocity of the blood flow.

If the gain is adjusted too large, overflow may likely occur. Data obtained from an analog to digital conversion circuit in a Doppler ultrasound system may have signal distortions in its frequency shift signal after Doppler signal analysis. Similarly, the content of the Doppler shift signal representing the velocity of the blood flow is also not reflected truly and completely.

In addition, in a general ultrasonic heart rate monitor, when Doppler ultrasound is used to measure the blood flow on the heart valve of the patient, it is generally required to transmit the Doppler signal received by the ultrasonic probe to a computer through a wired connection, such that the computer can calculate the velocity of the blood flow of the patient and draw an image of the velocity of the blood flow.

In other words, the existing ultrasonic heartbeat monitor may not be easily carried around because the area occupied by the ultrasonic probe is too large and it needs to be connected through a wired connection.

Therefore, there is a need for an ultrasound system that addresses the aforementioned issues in the prior art, in particular, a portable ultrasound system with high SNR that measures the velocity of the blood flow quickly and accurately.

### SUMMARY OF THE DISCLOSURE

The present disclosure provides an ultrasonic system with adaptive overflow and gain control. It uses two-stage gain controls to achieve high SNR and the correct analysis of results. The first stage adopts an analog gain, and monitors whether the signal may overflow or its signal strength is too weak and adjusts the gain value as large as possible without causing overflow in order to improve the SNR. The second stage adopts digital gain. Gains of all data in a Short Time Fourier Transform (STFT) data segment used in a Doppler signal analyzer are adjusted to be the same to ensure the correct result of STFT analysis. As a result, the medical ultrasound system proposed by the present disclosure is portable while capable of performing quick and accurate measurements (e.g., blood flow velocity) with a high SNR.

Thus, the present disclosure is to provide an ultrasound system with adaptive overflow and gain control, which may include: an analog gain filter configured for receiving and filtering ultrasound Doppler shift signal data, and amplifying the analog Doppler shift signal data to produce amplified analog Doppler shift signal data with a gain amplified; an analog-to-digital converter configured for converting the amplified analog Doppler shift signal data into amplified digital Doppler shift signal data; an adaptive gain control module configured for continuously monitoring the amplified digital Doppler shift signal data and subsequent amplified digital Doppler shift signal data, updating a gain of the analog gain filter using a first gain algorithm for the amplified digital Doppler shift signal data subsequently outputted by the analog-to-digital converter to fall within a predetermined range, and combining one or more amplified digital Doppler shift signal data and their gains into a set of amplified gain-containing digital Doppler shift signal data; and a digital gain module configured for sequentially receiving and storing the set of amplified gain-containing digital Doppler shift signal data and subsequent sets of amplified gain-containing digital Doppler shift signal data in a data buffer of the digital gain module, and sequentially retrieving from the data buffer, in chronological order, an amplified gain-containing data segment and their gains, and adjusting the amplified gain-containing data segment into a same-gain data segment having the same gain using a second gain algorithm,

The present disclosure is also to provide a method for controlling an ultrasound system with adaptive overflow and gain control, which may include: converting, by an analog-to-digital converter, amplified analog Doppler shift signal data into amplified digital Doppler shift signal data; updating, by an adaptive gain control module, the gain of an analog gain filter using an analog gain algorithm for the amplified digital Doppler shift signal data subsequently outputted by the analog-to-digital converter to fall within a predetermined range, and combining one or more amplified gain-containing digital Doppler shift signal data into a set of amplified gain-containing digital Doppler shift signal data; and sequentially receiving, by a digital gain module, the set of amplified gain-containing digital Doppler shift signal data and subsequent sets of amplified gain-containing digital Doppler shift signal data, sequentially retrieving from the received sets of amplified gain-containing digital Doppler shift signal data, in chronological order, a fixed amount of amplified gain-containing digital Doppler shift signal data as an amplified gain-containing data segment, and adjusting the amplified gain-containing data segment into a same-gain data segment having the same gain using a digital gain algorithm.

### BRIEF DESCRIPTION OF THE DRAWINGS

FIG. 1 is a block diagram depicting an ultrasound system with adaptive overflow and gain control in accordance with a first embodiment of the present disclosure;

FIG. 2 is a block diagram depicting an ultrasound system with adaptive overflow and gain control in accordance with a second embodiment of the present disclosure;

FIG. 3 is a schematic diagram illustrating adjusting of data to be within a predetermined range in accordance with the present disclosure;

FIG. 4 is a flowchart illustrating a first gain algorithm implemented in accordance with the present disclosure;

FIG. 5 is a schematic diagram illustrating transmission of a set of amplified digital Doppler shift signal data and their gains in a packet in accordance with the present disclosure;

FIG. 6 is a flowchart illustrating a first gain algorithm implemented in accordance with the present disclosure;

FIG. 7 is a block diagram depicting an ultrasound system with adaptive overflow and gain control in accordance with a third embodiment of the present disclosure;

FIG. 8 is a block diagram depicting an ultrasound system with adaptive overflow and gain control in accordance with a fourth embodiment of the present disclosure;

FIG. 9 is a flowchart illustrating an ultrasound method for adjusting gain control in accordance with the present disclosure;

FIG. 10A is an image depicting blood flow velocity when the gain is adjusted to be too small;

FIG. 10B is an image depicting blood flow velocity when the gain is appropriately adjusted;

FIG. 10C is an image depicting blood flow velocity when the gain is adjusted to be too large;

FIG. 11A corresponds to a software and hardware implementation of steps S8-S9 in FIG. 6; and

FIG. 11B corresponds to a software and hardware implementation of steps S10-S12 in FIG. 6.

### DETAILED DESCRIPTION

The present disclosure is described by the following specific embodiments. Those with ordinary skills in the arts can readily understand other advantages and functions of the present disclosure after reading the disclosure of this specification. The present disclosure may also be practiced or applied with other different implementations. Based on different contexts and applications, the various details in this specification can be modified and changed without departing from the spirit of the present disclosure.

Body fluid flow rate, including blood flow velocity, lymphatic fluid velocity etc., can generally be used as the basis for medical diagnosis. For example, blood flow velocity refers to the velocity of the flow of red blood cells in a blood vessel. It is a very important physiological parameter that reflects numerous body functions, such as heart function, circulatory system function and human metabolism level and the like. Therefore, the detection of human blood velocity has great physiological significance and clinical value in clinical diagnosis, operation monitoring and so on. Blood flow velocity can also help diagnose vascular diseases, such as peripheral vascular sclerosis, stenosis, obstruction, plaque assessment, etc. The blood flow velocity also has important clinical values in aspects such as in the determination of the replantation of severed limbs and vascular integrity of burn patients. It has become one of the important clinically diagnostic tools.

Therefore, in view of the above mentioned and other shortcomings of the general equipment for detecting human blood flow velocity, an ultrasound system with adaptive overflow and gain control is proposed.

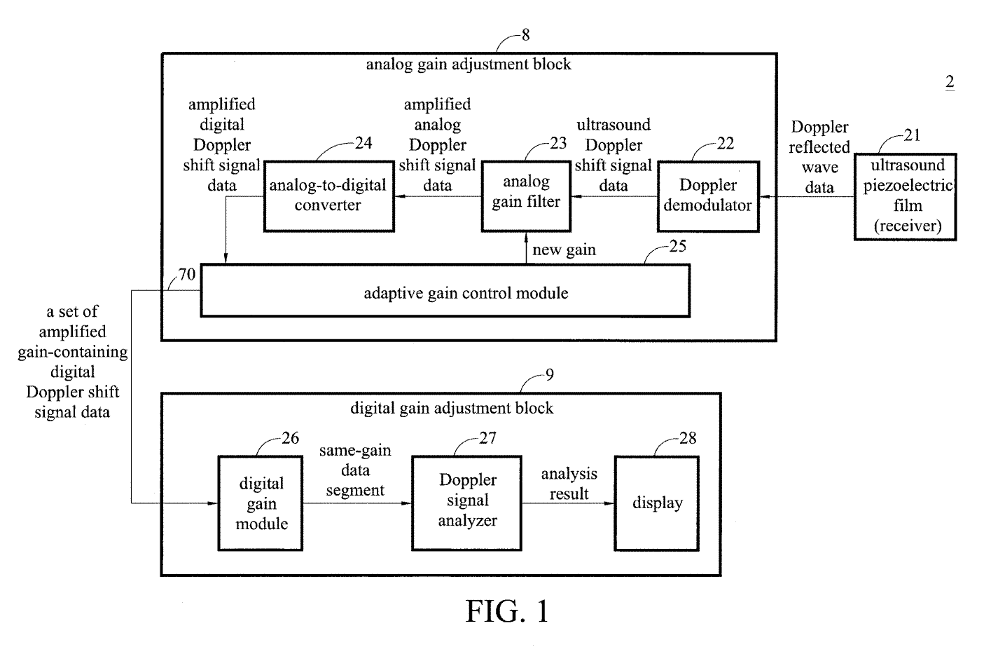

As shown in FIG. 1, a block diagram depicting an ultrasound system with adaptive overflow and gain control in accordance with the present disclosure is shown. It primarily includes two blocks for two-stage gain adjustments. The first stage uses an analog gain adjustment block 8 and the second stage uses a digital gain adjustment block 9. The analog gain adjustment block 8 receives signal data from an ultrasound piezoelectric film 21, and includes a Doppler demodulator 22, an analog gain filter 23, an analog-to-digital converter 24 and an adaptive gain control module 25. The digital gain adjustment block 9 includes a digital gain module 26, a Doppler signal analyzer 27 and a display 28.

The ultrasound piezoelectric film 21 first emits ultrasound of a particular frequency to a site to be detected (such as pulmonary artery blood vessels), and sequentially receives a Doppler reflective wave data using the Doppler Effect.

The Doppler demodulator 22 then sequentially demodulates the Doppler reflective wave data into ultrasound Doppler shift signal data.

The analog gain filter 23 then sequentially filters out high frequency noise signals from the ultrasound Doppler shift signal data and amplifies the weak ultrasound Doppler shift signal data. For example, assuming the maximum velocity of the human blood flow signal to be monitored is 1.2 m/s and the transmission frequency of the ultrasound is 2.5 MHz, by using a Doppler Effect equation, the maximum frequency of the received frequency shift about 4 KHz can be calculated. The filter can be set to filter out signals with frequencies higher than 4 KHz, while passing signals having frequencies below 4 KHz (including blood flow signals and noise below 4 KHz). Noise below 4 KHz in the system may enter through the ultrasound receiver, as well as other circuits, such as a speaker and a Bluetooth transmission module. Therefore, under the condition that data overflow will not occur, the gain is increased as high as possible to lower the ratio of noise from other circuits and increase the SNR (signal-to-noise ratio). As a result, amplified analog Doppler shift signal data is produced by the analog gain filter 23. It goes without saying that the above parameters are all doubled if it is assumed that the maximum velocity of the human blood flow signal to be monitored is 2.4 m/s.

The analog gain filter 23 can be implemented by using independent integrated circuits, modules, electronic components, or a combination of the integrated circuits, modules and electronic components. For example, under some circumstances, one existing analog gain filter module may be sufficed. However, if the range of gain that can be adjusted by the analog gain filter module is too narrow, a resistor gain circuit (not shown) consisting of some resistors and variable resistors can be added to the front of a signal input of the analog gain filter module 23, such that incoming signals are first amplified in the resistor gain circuit and then sent to the analog gain filter module 23. However, the ways in which signals are amplified are not limited to those described above.

The analog-to-digital converter 24 then sequentially converts the amplified analog Doppler shift signal data into an amplified digital Doppler shift signal data.

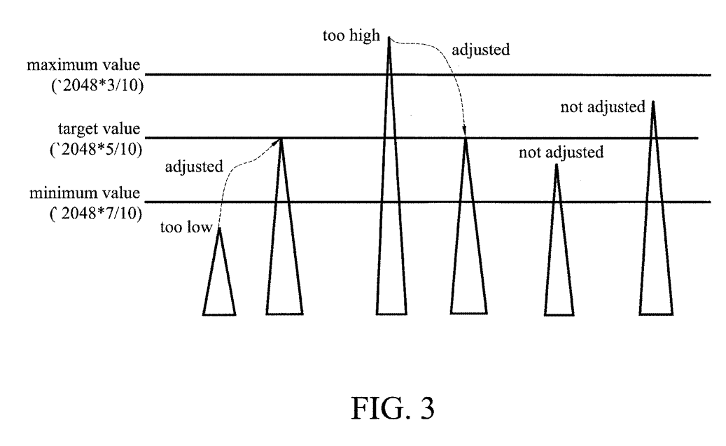

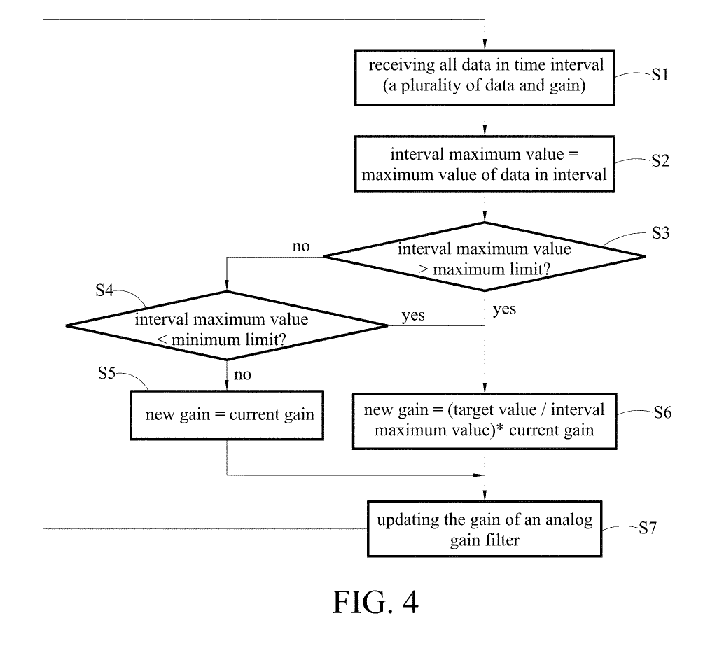

Now refer to FIG. 3 for more details of the adaptive gain control module 25. When a set of amplified digital Doppler shift signal data outputted by the analog-to-digital converter 24 is not within (e.g., higher than or lower than) a predetermined range (2048×3/10=615−2048×7/10=1434), the adaptive gain control module 25 performs a first gain algorithm (as shown in steps S1 to S7 in FIG. 4), such that subsequent sets of amplified digital Doppler shift signal data will be adjusted back to be within the predetermined range. The adaptive gain control module 25 sequentially receives the digital Doppler shift signal data outputted by the analog-to-digital converter 24, and performs the first gain algorithm (an analog gain algorithm) shown in the flowchart of FIG. 4, including steps S1-S7 below:

Step S1: All data and gains within a time interval are received, i.e., a plurality of data and gains are received. In other words, all these data and gains refer to an amplified digital Doppler shift signal data and subsequent amplified digital Doppler shift signal data sequentially received by the adaptive gain control module 25 from the analog-to-digital converter 24. For example, if the time interval for adjusting gain is set to 2 seconds, and the sampling rate per second is 8 KHz, the number of data received in the 2 seconds will be 16,000.

Step S2: An interval maximum value is defined from these data in the time interval. In other words, the interval maximum value=the maximum value of all data in the time interval. In other words, an interval maximum value is defined from all the data and gains, which are the amplified digital Doppler shift signal data and the subsequent amplified digital Doppler shift signal data sequentially received by the adaptive gain control module 25 from the analog-to-digital converter 24. In this embodiment, the maximum value of the 16,000 data will be the interval maximum value.

Step S3: It is determined whether the interval maximum value is greater than a maximum limit. If the interval maximum value is not greater than the maximum limit (no), proceed to step S4. If the interval maximum value is greater than the maximum limit (yes), step S6 is performed. For example, the analog-to-digital converter 24 uses a 12-bit resolution converter, and the numerical range is between −2¹¹(−2048) and +2¹¹(+2048) with a center value of 0. Suppose that the interval maximum value (must be a positive value) is set to be controlled between 2048×7/10=1434 and 2048×3/10=615. If the interval maximum value is greater than 1434, subsequent data may have overflowed; on the other hand, if the interval maximum value is less than 615, the SNRs of subsequent data may be too weak. In both cases, a correct signal cannot be shown. At this time, the maximum limit (positive maximum limit) is 1434, and the minimum limit (positive minimum limit) is 615. Assume, in this example, that the maximum value of 16,000 data received in the two seconds is 300 (the interval maximum value=300), which is less than the maximum limit 1434. Thus, proceed to step S4.

Step S4: It is determined whether the interval maximum value is less than a minimum limit. If the interval maximum value is not less than a minimum limit (no), proceed to step S5. If the interval maximum value is less than the minimum limit (yes), step S6 is performed. In this example, the maximum value (300) of 16,000 data received in the two seconds is less than the minimum limit (615). Thus, proceed to step S6.

Step S5: The gain of the analog gain filter 23 is not updated. Therefore, the new gain equals to the current gain of the analog gain filter 23 (new gain=the current gain).

Step S6: The gain is updated. The new gain equals to target value divided by interval maximum value and then multiplied by current (gain new gain=(target value/interval maximum value)×current gain). When the gain needs to be adjusted, regardless of whether the interval maximum value was too large or too small, the interval maximum value is adjusted to a target data value. The target data value is between the maximum limit and the minimum limit. In this example, the target data value is set to be the middle value between the maximum limit and the minimum limit, i.e., target data value=2048×5/10=1024. Assume that the gain of all the data in an interval is 3 (the gains of data in the same time interval will not be changed), and the new gain is updated to 3×615/300=6 (should be 6.15, but round to the nearest whole number).

Step S7: The gain of the analog gain filter 23 is updated to the new gain obtained in step S6. Once the adaptive gain control module 25 calculates a new gain, the new gain is set in the analog gain filter 23. As a result, the maximum value of all amplified data (all amplified digital Doppler shift signal data) in a subsequent interval would be between the predetermined range defined by the maximum limit and the minimum limit.

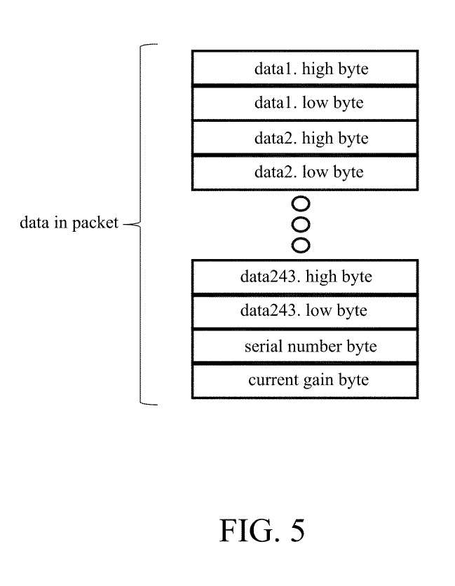

Regardless of whether step S5 (no change to the gain) or step S7 (the gain is updated) is performed based on the results of steps S3 and S4, the adaptive gain control module 25 will transmit a set of amplified gain-containing digital Doppler shift signal data to the digital gain module 26. See FIG. 5, which is a schematic diagram depicting data bytes of 243 data (associated with blood flow velocity of the pulmonary artery) plus a serial number byte and a current gain byte. The set of amplified gain-containing digital Doppler shift signal data is implemented as a packet and transmitted to the digital gain module 26.

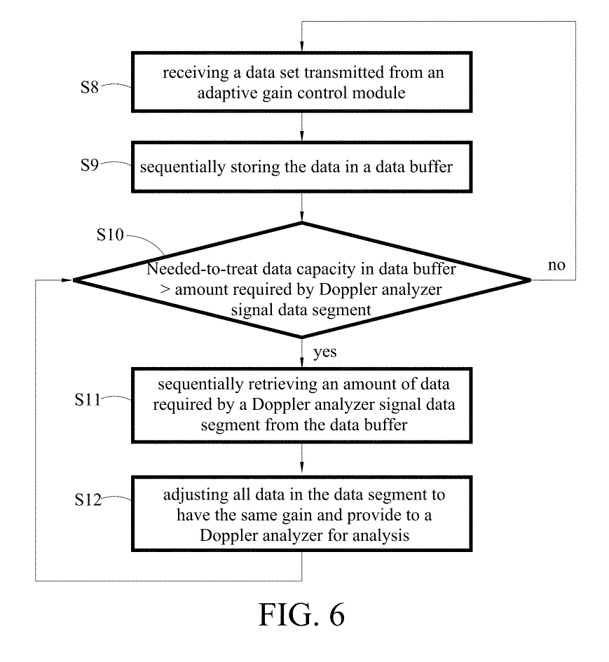

The digital gain module 26 may receive the set of amplified gain-containing digital Doppler shift signal data and subsequent sets of amplified gain-containing digital Doppler shift signal data via Bluetooth Low Energy (BLE) 70 or other communication methods, and successively extract, in chronological order, an amplified gain-containing data segment from all the sets of the amplified gain-containing digital Doppler shift signal data. Thereafter, according to a second gain algorithm (a digital gain algorithm) shown in steps S8-S12 in a flowchart of FIG. 6, the amplified gain-containing data segment is converted into a same-gain data segment with the same gain, and this same-gain data segment is provided to the Doppler signal analyzer 27 for analysis.

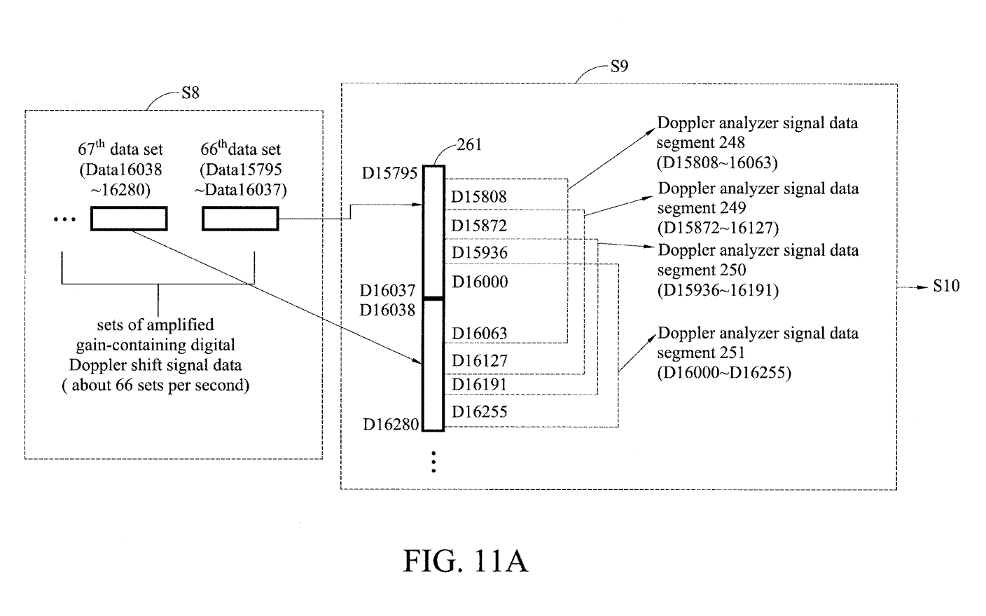

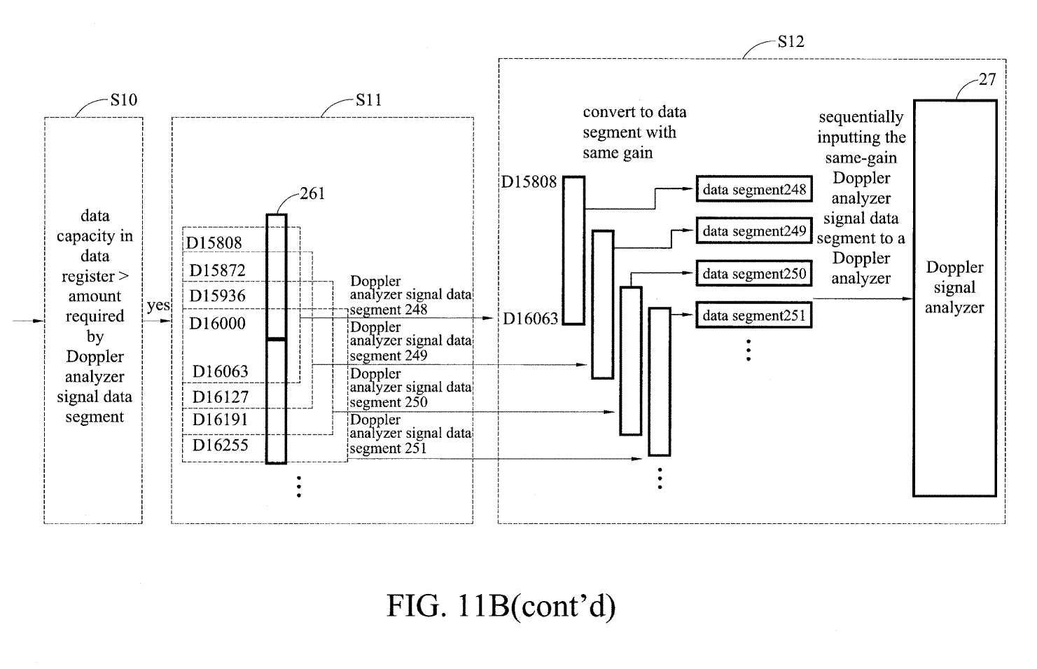

The second gain algorithm (a digital gain algorithm) in the flowchart of FIG. 6 includes steps S8-S12, and FIG. 11A is referred to at the same time, which is a software and hardware implementation of steps S8-S9.

Step S8: Data sets (each of the data set includes one or a plurality of data and the gain) are received from the adaptive gain control module 25. The data sets refer to a set of amplified gain-containing digital Doppler shift signal data and the subsequent sets of amplified gain-containing digital Doppler shift signal data. For example, the adaptive gain control module 25 transmits the set of amplified gain-containing digital Doppler shift signal data, as shown in FIG. 5. Each of the data set includes 243 amplified data and the gain information. Assuming after measurement has started, 66 sets of data had already been successively received (each data set includes 243 data and their gain), now the 67^{th} data is received shown in FIG. 11A.

Step S9: These data sets (also the set of amplified gain-containing digital Doppler shift signal data and subsequent sets of amplified gain-containing digital Doppler shift signal data) are sequentially stored in a data buffer 261 in the digital gain module 26 shown in FIG. 11A. The Nth set of amplified gain-containing digital Doppler shift signal data is stored in the <start position ((N−1)×243), end position ((N−1)×243+242)> of the data buffer 261.

In this example, as 66 data sets received previously have already been stored in the data buffer 261, these data sets occupy position 0 to position 16037 (66×243=16038) of the data buffer 261, wherein the 66^{th} set of amplified gain-containing digital Doppler shift signal data is stored in <15795, 16037> of the data buffer 261, and the 67^{th} set of amplified gain-containing digital Doppler shift signal data is stored in <16038, 16280> of the data buffer 261.

FIG. 11B is referred to at the same time, which is a software and hardware implementation of steps S10-S12.

Step S10: Referring to FIG. 6 and FIG. 11B, it is determined if the needed-to-treat data capacity of the data buffer 261 is greater than the amount of space needed by the data segments to be analyzed by the Doppler signal analyzer 27. If the data capacity of the data buffer 261 is greater than the amount of space needed by the data segments (yes), proceed to step S11; if the data capacity of the data buffer 261 is not greater than the amount of space needed by the data segments (no), proceed to step S8. For example, the Doppler signal analyzer 27 may perform data analysis using Short Time Fourier Transform (STFT). The Doppler signal analyzer 27 receives equivalent gain data segments based on chronological order, and sequentially analyzes each data segment.

Assume that the size of data segment required by the Doppler signal analyzer 27 is 256 data, and each time the Doppler signal analyzer 27 moves 64 data to obtain the next segment of 256 data. Using mathematical representation, <initial data position, end data position> of N data segment is <(N−1)×64, (N−1)×64+255>. From the above, it can be seen that the previous 66 data sets have provided 247 data segments (247−1)×64+255=15999, leaving with a total of 230 remaining data from position (248−1)×64=15808 to position 16037 (the size of data segment required by the Doppler signal analyzer 27 is 256 data), adding the 67^{th} data set received this time, there are a total of 473 (230+243) available data, which is greater than 256 data. Thus, step S11 is performed.

Step S11: Data segments required for the Doppler signal analysis are sequentially retrieved from the data buffer 261. In this example (e.g., in FIG. 11B), when the 67^{th} packet is received, four data segments (data segments 248-251) are retrieved for STFT analysis. The <initial position, end position> of these four data segments are <15808, 16063>, <15872, 16127>, <15936, 16191>and <16000, 16255>, respectively.

Step S12: All the data in a data segment are adjusted to the same gain and are provided to the Doppler signal analyzer 27 for analysis. Before a data segment is provided for STFT analysis, all the data in the data segment are converted to numerical values by using the same gain. Assume that the previous 66 data sets were in the same time interval (the gains of data in the same time interval will not be changed) with a data gain of 3, and the previous 247 data segments do not require numerical value adjustment before STFT analysis. Assume that the 67^{th} data set belongs to another time interval with a gain adjusted to 6, and in the data segments 248-251, data in the same data segment may have different gain, some may have a gain of 3, while some may have a gain of 6. Therefore, the gains need to be adjusted, and the data updated to new numerical values before STFT analysis can be performed.

For example, assume that the gains of the data segment are determined to be adjusted to 3, and a data in this data segment has a value of N1 and a gain of 3. This data does not need to be adjusted; on the other hand, if there is another data in this data segment with a value of M1 and a gain of 6, this data will need to be updated to M2=M1×3/6=0.5 M1.

Once all the data in the data segment have the same gain, the digital gain module 26 can then provide them to the Doppler signal analyzer 27, in which the STFT is performed to convert them into an image for medical diagnosis (i.e., analysis result) to be shown on the display 28.

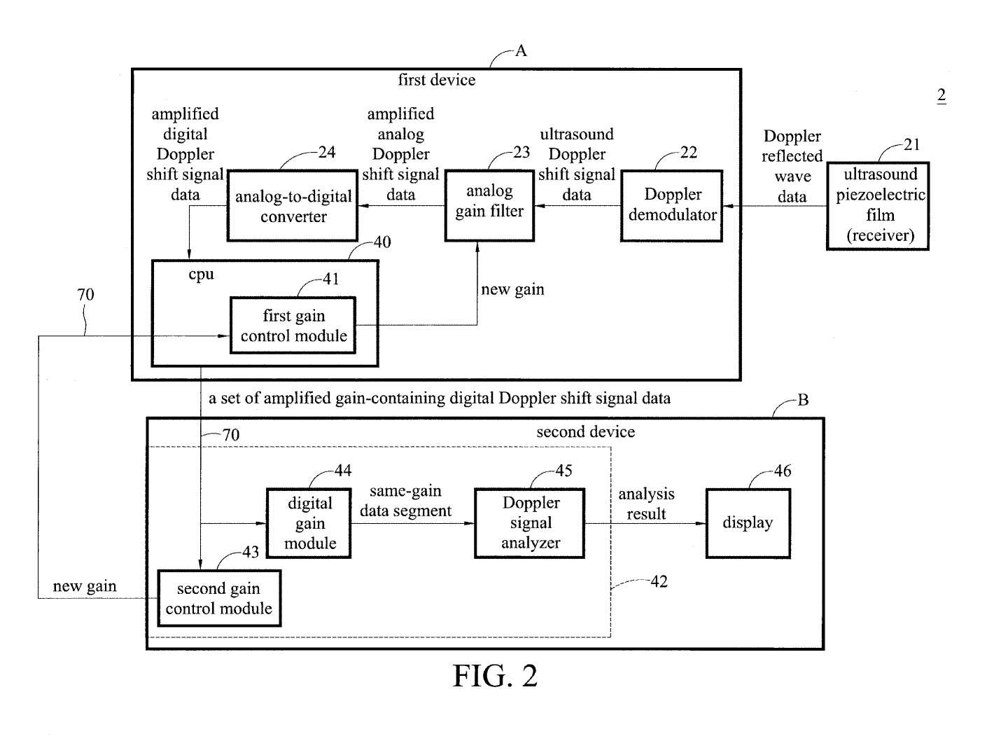

Similar to FIG. 1, FIG. 2 is a block diagram depicting an ultrasound system with adaptive overflow and gain control in accordance with a second embodiment of the present disclosure.

An ultrasound system 2 with adaptive overflow and gain control includes two blocks, i.e., a first device A and a second device B. The first device A is, for example, an embedded system, including the Doppler demodulator 22, the analog gain filter 23, the analog-to-digital converter 24, and a first gain control module 41 included in a central processing unit (CPU) 40. The CPU 40 sequentially receives amplified digital Doppler shift signal data converted by the analog-to-digital converter 24, and combines a plurality of these amplified digital Doppler shift signal data with their gains into a set of amplified gain-containing digital Doppler shift signal data, and then transmits this set of amplified gain-containing digital Doppler shift signal data to the second device B via the BLE 70. The CPU 40 also receives a new gain command from the second device B via the BLE 70 (wherein the new gain command is calculated by a second gain control module 43 in another CPU 42), such that when the first gain control module 41 of the CPU 40 starts collecting data of a new data set, the new gain command is set to change the gain of the analog gain filter 23.

The second device B is, for example, a smartphone, and the CPU 42 includes the second gain control module 43 (a type of analog gain control module), a digital gain module 44, a Doppler signal analyzer 45, and a display 46 included in the second device B for displaying the results of the analysis (e.g., images for medical diagnosis). The CPU 42 of the second device B sequentially receives a set of amplified gain-containing digital Doppler shift signal data from the first device A, and the second gain control module 43 performs the first gain algorithm (illustrated in steps S1-S7 of FIG. 4) after a time interval to calculate a new gain command, which is sent to the first gain control module 41 of the CPU 40 via BLE 70. The first gain control module 41 then sends the new gain to the analog gain filter 23 for changing the gain of the analog gain filter 23, such that a subsequent set of amplified digital Doppler shift signal data is adjusted back to be within a predetermined range. The function of the adaptive gain control module 25 in FIG. 1 is achieved by the first gain control module 41 and the second gain control module 43 in collaboration in FIG. 2. In other words, the adaptive gain control module 25 of FIG. 1 is divided into the first gain control module 41 and the second gain control module 43 of FIG. 2.

The digital gain module 44 sequentially receives the set of amplified gain-containing digital Doppler shift signal data and subsequent sets of amplified gain-containing digital Doppler shift signal data, uses the second gain algorithm (as illustrated in steps S8-S12 of FIG. 6 above) to store all the data received in the data buffer 261 in chronological order, sequentially retrieves amplified gain-containing data segment(s) in chronological order, and converts the amplified gain-containing data segments into a same-gain data segment with the same gain. In other words, converting the amplified gain-containing data segments into a same-gain data segment with the same gain means that the set of amplified gain-containing digital Doppler shift signal data and the subsequent sets of amplified gain-containing digital Doppler shift signal data are all adjusted to have the same gain. Thereafter, the same-gain data segments are sequentially provided to the Doppler signal analyzer 45 for analysis.

The Doppler signal analyzer 45 converts each segment of digital signal data with the same gain into an image (analysis result) for medical analysis according to Short Time Fourier Transform to be displayed on the display 46.

The CPU 40 is provided in the embedded system, while the CPU 42 is provided on the smartphone, wherein the second gain control module 43 is embedded in the CPU 42 for performing the first gain algorithm. The main purpose of this is so that the CPU 42 can speed up the execution of the first gain algorithm and reduce computational costs of the CPU 40 in the first device A. As a result, the size of the first device A can be reduced.

Similar to FIGS. 1 and 2, FIG. 7 shows a block diagram depicting an ultrasound system with adaptive overflow and gain control 2 in accordance with a third embodiment of the present disclosure.

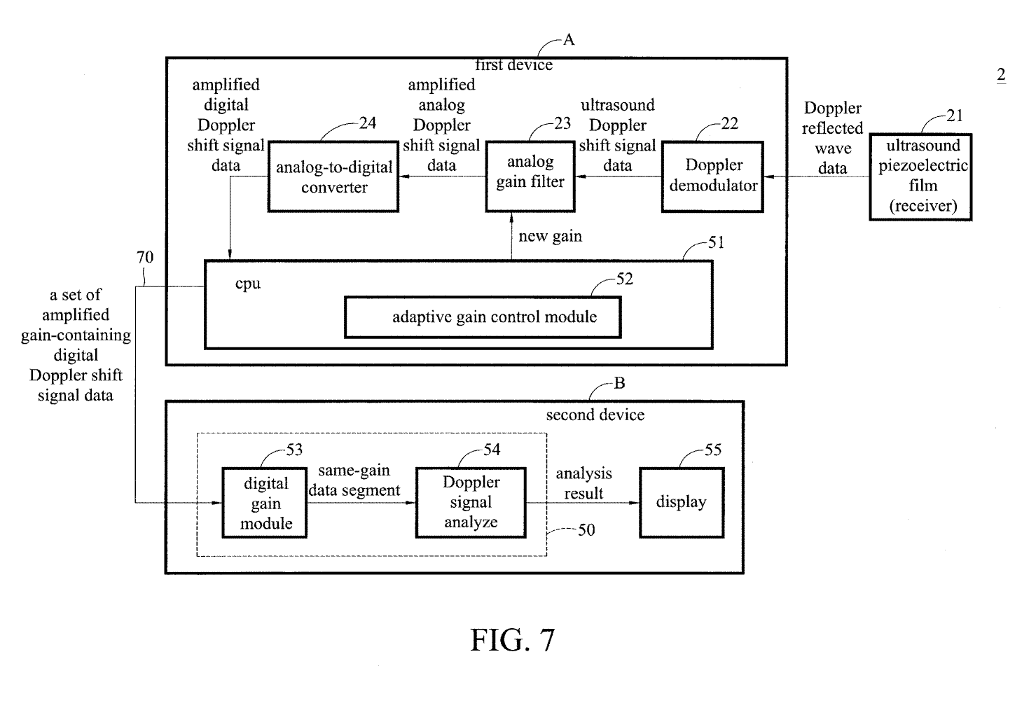

An adaptive gain control module 52 is embedded in a CPU 51. The performance efficiency of the first gain algorithm (as shown in steps S1-S7 of FIG. 4) can be speed up by the CPU 51. A digital gain module 53 and a Doppler signal analyzer 54 in FIG. 7 are embedded in a CPU 50. Similarly, the performance efficiency of the second gain algorithm (as shown in steps S8-S12 of FIG. 6) can be speed up by the CPU 50. The functions of these modules as well as a display 55 are similar to those described with respect to FIG. 1, further description thereof hereby omitted.

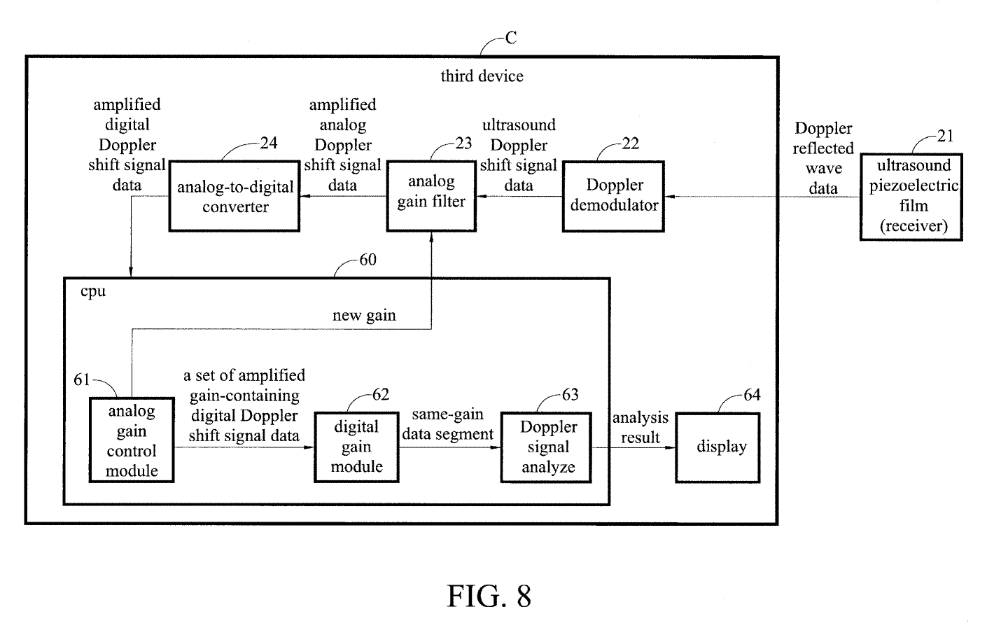

FIG. 8 is a block diagram depicting an ultrasound system with adaptive overflow and gain control in accordance with a fourth embodiment of the present disclosure.

The ultrasound system 2 with adaptive overflow and gain control is embedded in a third device C (e.g., a portable or desktop ultrasound device), and a CPU 60 includes an analog gain control module 61 for performing the first gain algorithm (as shown in steps S1-S7 of FIG. 4), a digital gain module 62 for performing the second gain algorithm (as shown in steps S8-S12 of FIG. 6), a Doppler signal analyzer 63, and a display 64 for showing the result of analysis. The functions of these modules are similar to those described with respect to FIG. 1, and will not be repeated.

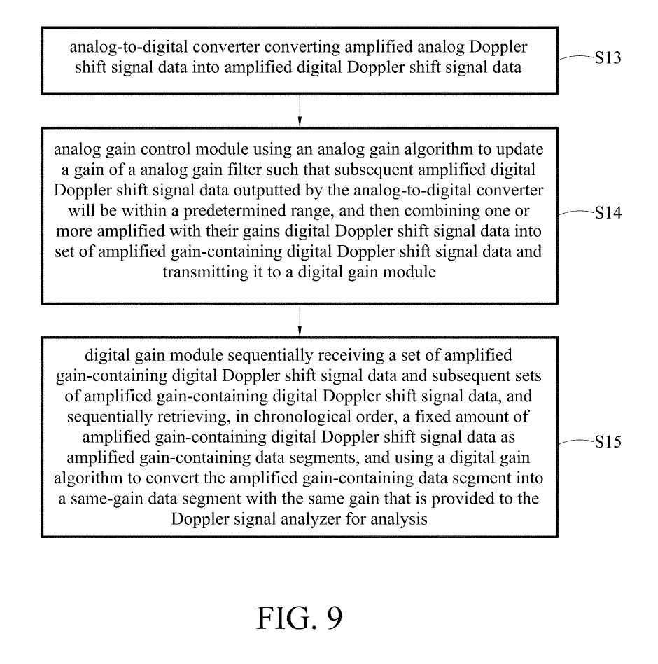

FIG. 9 is a flowchart illustrating steps of an ultrasound method for adjusting gain control in accordance with the present disclosure, and is realized with some of the components of the system in FIG. 8. The steps include the following.

Step S13: The analog-to-digital converter 24 converts an amplified analog Doppler shift signal data into an amplified digital Doppler shift signal data.

Step S14: The analog gain control module 61 (or adaptive gain control module) uses an analog gain algorithm (steps S1-S7 of FIG. 4) to update the gain of the analog gain filter 23, such that the subsequent amplified digital Doppler shift signal data outputted by the analog-to-digital converter 24 will be within a predetermined range. Then, one or more amplified digital Doppler shift signal data with their gains (one or more amplified gain-containing digital Doppler shift signal data) are combined into a set of amplified gain-containing digital Doppler shift signal data, and this set of amplified gain-containing digital Doppler shift signal data is transmitted to the digital gain module 62.

Step S15: The digital gain module 62 sequentially receives the set of amplified gain-containing digital Doppler shift signal data and subsequent sets of amplified gain-containing digital Doppler shift signal data, and sequentially retrieves, in chronological order, a fixed amount of the amplified gain-containing digital Doppler shift signal data as an amplified data segment and their gains. Then, a digital gain algorithm (steps S8-S12 of FIG. 6) is used to convert the amplified gain-containing data segment into a same-gain data segment with the same gain. The same-gain data segment is provided to the Doppler signal analyzer 63 for analysis.

In conclusion, the ultrasound system and the ultrasound method with adaptive overflow and gain control are provided by the present disclosure. The first stage adopts an analog gain. At this stage, signals are monitored to see if they are higher or lower than a predetermined range, and the gain is adjusted to be as large as possible without causing overflow, such that SNR is improved. The second stage adopts a digital gain, in which the gains of all the data in the same segment required for Doppler signal analyzer are adjusted to be the same to facilitate STFT. As a result, the medical ultrasound system of the present disclosure is portable while capable of performing quick and accurate measurements (e.g., blood flow velocity) with a high SNR.

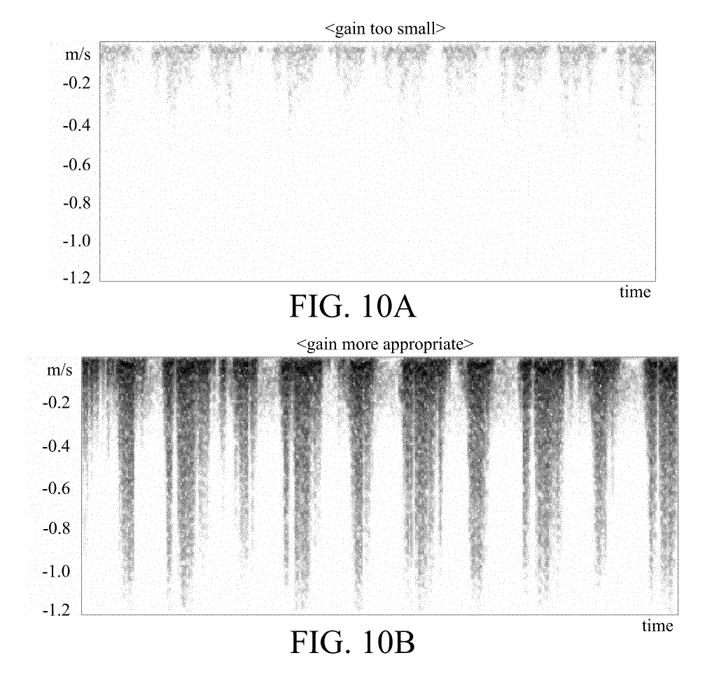

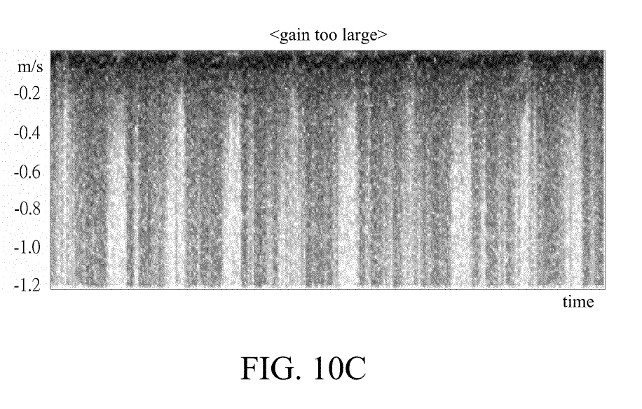

FIG. 10A is an image depicting blood flow velocity when the gain is adjusted to be too small. FIG. 10A demonstrates that low gain will results in poor SNR, and the contents of the Doppler shift signals representing the blood flow velocity cannot be accurately and fully shown.

FIG. 10C is an image depicting blood flow velocity when the gain is adjusted to be too large. FIG. 10C demonstrates that when the gain is too high, signal distortions will occur in the shift signals after STFT conversion from Doppler digital signals converted by the analog-to-digital conversion circuit in the Doppler ultrasound system. Similarly, the contents of the Doppler shift signals representing the blood flow velocity cannot be accurately and fully shown.

On the other hand, according to the ultrasound system and the ultrasound method with adaptive overflow and gain control proposed by the present disclosure, an image depicting blood flow velocity when the gain is appropriate is shown in FIG. 10B. Since an analog gain is adopted in the first stage and a digital gain is adopted in the second stage, SNR is increased, and the data in the data segments entering the STFT all have the same gain, correct blood flow information can be obtained from performing STFT continuously, and a satisfactory image with better contrast can be displayed on the display for medical diagnosis.

The above embodiments are only used to illustrate the principles of the present disclosure, and should not be construed as to limit the present disclosure in any way. The above embodiments can be modified by those with ordinary skill in the art without departing from the scope of the present disclosure as defined in the following appended claims.

What is claimed is:

1. An ultrasound system, comprising:
an analog gain filter configured for receiving and filtering ultrasound Doppler shift signal data, and amplifying the ultrasound Doppler shift signal data to produce amplified analog Doppler shift signal data with a gain amplified;
an analog-to-digital converter configured for converting the amplified analog Doppler shift signal data into amplified digital Doppler shift signal data;
an adaptive gain control module configured for continuously monitoring the amplified digital Doppler shift signal data and subsequent amplified digital Doppler shift signal data, updating a gain of the analog gain filter by using a first gain algorithm for the amplified digital Doppler shift signal data subsequently outputted by the analog-to-digital converter to fall within a predetermined range, and combining one or more amplified digital Doppler shift signal data and their gains into a set of amplified gain-containing digital Doppler shift signal data; and
a digital gain module configured for sequentially receiving and storing the set of amplified gain-containing digital Doppler shift signal data and subsequent sets of amplified gain-containing digital Doppler shift signal data in a data buffer of the digital gain module, and sequentially retrieving from the data buffer, in chronological order, an amplified gain-containing data segment and their gains, and adjusting the amplified gain-containing data segment into a same-gain data segment having the same gain using a second gain algorithm.

2. The ultrasound system of claim 1, wherein the adaptive gain control module is divided into a first gain control module and a second gain control module, the second gain control module performs the first gain algorithm for the amplified digital Doppler shift signal data subsequently outputted by the analog-to-digital converter to fall within the predetermined range, and the first gain control module updates the gain of the analog gain filter with a new gain generated by the second gain control module via the first gain algorithm.

3. The ultrasound system of claim 1, wherein the set of amplified gain-containing digital Doppler shift signal data is transmitted to the digital gain module via Bluetooth Low Energy (BLE) or other wireless or wired communication methods.

4. The ultrasound system of claim 1, further comprising a Doppler signal analyzer configured for sequentially converting the same-gain data segment and subsequent same-gain data segments into pictures, images or data for medical diagnosis.

5. The ultrasound system of claim 4, wherein the Doppler signal analyzer and the digital gain module are embedded in a smartphone.

6. The ultrasound system of claim 1, further comprising a Doppler demodulator configured for demodulating Doppler reflective wave data into the ultrasound Doppler shift signal data.

7. The ultrasound system of claim 1, wherein the second gain algorithm adjusts the amplified gain-containing data segment into the same-gain data segment having the same gain by adjusting the gains of the set of amplified gain-containing digital Doppler shift signal data and the subsequent sets of amplified gain-containing digital Doppler shift signal data to have the same gain.

8. The ultrasound system of claim 1, wherein the ultrasound Doppler shift signal data is generated by reflection from at least one site to be detected, and the at least one site to be detected is a body part of an animal.

9. The ultrasound system of claim 8, wherein the body part has a body fluid flow rate as a blood flow velocity or a lymphatic fluid velocity.

10. The ultrasound system of claim 1, which is integrated in a portable or desktop ultrasound device.

11. The ultrasound system of claim 1, wherein when the amplified digital Doppler shift signal data outputted by the analog-to-digital converter is not fallen within the predetermined range, the amplified digital Doppler shift signal data is adjusted back to be within the predetermined range.

12. A method for controlling an ultrasound system, comprising:
converting, by an analog-to-digital converter, amplified analog Doppler shift signal data into amplified digital Doppler shift signal data;
updating, by an adaptive gain control module, a gain of an analog gain filter using an analog gain algorithm for the amplified digital Doppler shift signal data subsequently outputted by the analog-to-digital converter to fall within a predetermined range, and combining one or more amplified gain-containing digital Doppler shift signal data into a set of amplified gain-containing digital Doppler shift signal data; and
sequentially receiving, by a digital gain module, the set of amplified gain-containing digital Doppler shift signal data and subsequent sets of amplified gain-containing digital Doppler shift signal data, sequentially retrieving from the received sets of amplified gain-containing digital Doppler shift signal data, in chronological order, a fixed amount of amplified gain-containing digital Doppler shift signal data as an amplified gain-containing data segment, and adjusting the amplified gain-containing data segment into a same-gain data segment having the same gain using a digital gain algorithm.

13. The method of claim 12, wherein the set of amplified gain-containing Doppler shift signal data is associated with an ultrasound Doppler shift signal data reflected from at least one site to be detected.

14. The method of claim 12, wherein the digital gain algorithm is executed to perform:
step S1: receiving the amplified digital Doppler shift signal data and the subsequent amplified digital Doppler shift signal data in a time interval;
step S2: defining an interval maximum value among the amplified digital Doppler shift signal data and the subsequent amplified digital Doppler shift signal data in a time interval;
step S3: determining whether the interval maximum value is greater than a maximum limit; and proceeding to step S4, if the interval maximum value is not greater than the maximum limit, proceeding to step S6;
step S4: determining whether the interval maximum value is less than minimum limit;
and proceeding to step S5, if the interval maximum value is not less than minimum limit, proceeding to step S6;
step S5: not updating the gain of the analog gain filter;
step S6: updating the gain to be a new gain=(a target value/the interval maximum value)×a current gain; and
step S7: updating the gain of the analog gain filter based on the new gain obtained in step S6.

15. The method of claim 12, wherein the digital gain algorithm is executed to perform:
step S8: receiving the set of amplified gain-containing digital Doppler shift signal data and the subsequent sets of amplified gain-containing digital Doppler shift signal data;
step S9: sequentially storing the set of amplified gain-containing digital Doppler shift signal data and the subsequent sets of amplified gain-containing digital Doppler shift signal data into a data buffer;
step S10: determining whether a data capacity of the data buffer is greater than an amount of space in a data segment required by a Doppler signal analyzer; proceeding to step S11, if the data capacity of the data buffer is not greater than the amount of data, returning to step S8;
step S11: sequentially retrieving the amount of data for the data segment required by the Doppler signal analyzer; and
step S12: adjusting the data in the data segment to be a same-gain data segment and providing the same-gain data segment to the Doppler signal analyzer for analysis.
